(19) United States
(12) Patent Application Publication
(10) Pub. No.: US 2021/0282687 A1
(43) Pub. Date: Sep. 16, 2021

(54) COGNITIVE ABILITY DETECTION APPARATUS AND COGNITIVE ABILITY DETECTION SYSTEM

(71) Applicant: Murata Manufacturing Co., Ltd., Kyoto (JP)

(72) Inventor: Hiroshi KUNIMATSU, Kyoto (JP)

(21) Appl. No.: 17/336,449

(22) Filed: Jun. 2, 2021

Related U.S. Application Data
(63) Continuation of application No. PCT/JP2019/050450, filed on Dec. 24, 2019.

(30) Foreign Application Priority Data
Dec. 27, 2018 (JP) 2018-244616

(51) Int. Cl. A61B 5/18; A61B 5/16; A61B 5/378; A61B 5/00; A61B 5/375; B60K 35/00

(52) U.S. Cl. CPC A61B 5/18; A61B 5/163; A61B 5/378; B60K 2370/149; A61B 5/375; B60K 35/00; B60K 2370/152; A61B 5/6803

(57) ABSTRACT

A cognitive ability detection apparatus (10) includes a visual information acquisition unit (12), a brain signal acquisition unit (13), and a detection unit (14). The visual information acquisition unit (12) is configured to acquire visual information of a subject to a visual stimulus included in video. The brain signal acquisition unit (13) is configured to acquire a brain signal of the subject. The detection unit (14) is configured to detect an event-related potential from the brain signal by using, as a starting point, a video trigger based on an occurrence timing of the visual stimulus, detect whether there is a point of gaze for the visual stimulus from the visual information, and detect a cognitive ability level to the visual stimulus.

### CROSS REFERENCE TO RELATED APPLICATION

This is a continuation of International Application No. PCT/JP2019/050450 filed on Dec. 24, 2019 which claims priority from Japanese Patent Application No. 2018-244616 filed on Dec. 27, 2018. The contents of these applications are incorporated herein by reference in their entireties.

### BACKGROUND OF THE DISCLOSURE

### Field of the Disclosure

This disclosure relates to a cognitive ability detection apparatus and a cognitive ability detection system that detect a cognitive ability to a visual stimulus.

### Description of the Related Art

Patent Document 1 discloses an unconscious learning method that uses neurofeedback. According to the unconscious learning method described in Patent Document 1, brain waves are measured while a subject is listening to the sound that serves as a task to be learned. In this unconscious learning method, learning is repeatedly performed based on brain waves.

Patent Document 1: Japanese Patent No. 6362332

### BRIEF SUMMARY OF THE DISCLOSURE

However, no technique for detecting a cognitive ability of a subject to a visual stimulus is present in the related art including the technique of Patent Document 1.

Accordingly, it is an object of the present disclosure to provide a cognitive ability detection technique that enables the detection of a cognitive ability of a subject to a visual stimulus.

A cognitive ability detection apparatus according to this disclosure includes a visual information acquisition unit, a brain signal acquisition unit, and a detection unit. The visual information acquisition unit is configured to acquire visual information of a subject to a visual stimulus included in video. The brain signal acquisition unit is configured to acquire a brain signal of the subject. The detection unit is configured to detect an event-related potential from the brain signal by using, as a starting point, a video trigger based on an occurrence timing of the visual stimulus, detect whether there is a point of gaze for the visual stimulus from the visual information, and detect a cognitive ability level to the visual stimulus.

In this configuration, a cognitive reaction of the subject to the visual stimulus is acquired based on the visual information and the brain signal. Therefore, a detection level of a cognitive ability to the visual stimulus is improved compared with the case where the visual information alone is used or where the brain signal alone is used.

According to the present disclosure, a cognitive ability of a subject to a visual stimulus is successfully detected.

### BRIEF DESCRIPTION OF THE SEVERAL VIEWS OF THE DRAWINGS

FIG. 1 is a functional block diagram of a cognitive ability detection system including a cognitive ability detection apparatus according to a first embodiment of the present disclosure.

FIG. 2 is a functional block diagram illustrating a configuration of a brain signal acquisition unit.

FIG. 3 is a functional block diagram illustrating a configuration of a detection unit.

FIG. 4 is a diagram illustrating an overview of a state of a detection gear worn by a subject whose cognitive ability is to be detected.

FIG. 5 is a functional block diagram illustrating a configuration of a visual stimulus presentation apparatus.

FIG. 6 is a diagram illustrating a concept of cognitive ability detection.

FIG. 7 is a flowchart of a cognitive ability detection method using P300 according to the first embodiment of the present disclosure.

FIG. 8 is a flowchart of a cognitive ability detection method using P300 and ERN according to the first embodiment of the present disclosure.

FIG. 9 is a flowchart illustrating an example of a cognitive ability detection method using ERN.

FIG. 10 is a functional block diagram of a cognitive ability detection system including a cognitive ability detection apparatus according to a second embodiment of the present disclosure.

FIG. 11 is a functional block diagram illustrating a configuration of a detection unit.

FIG. 12 is a diagram illustrating a concept of cognitive ability detection.

FIG. 13 is a flowchart of a cognitive ability detection method using P300, ERN, and FRN according to the second embodiment of the present disclosure.

FIG. 14 is a flowchart of an example of a cognitive ability detection method using FRN.

FIG. 15 is a diagram illustrating a concept of cognitive ability detection.

### DETAILED DESCRIPTION OF THE DISCLOSURE

### First Embodiment

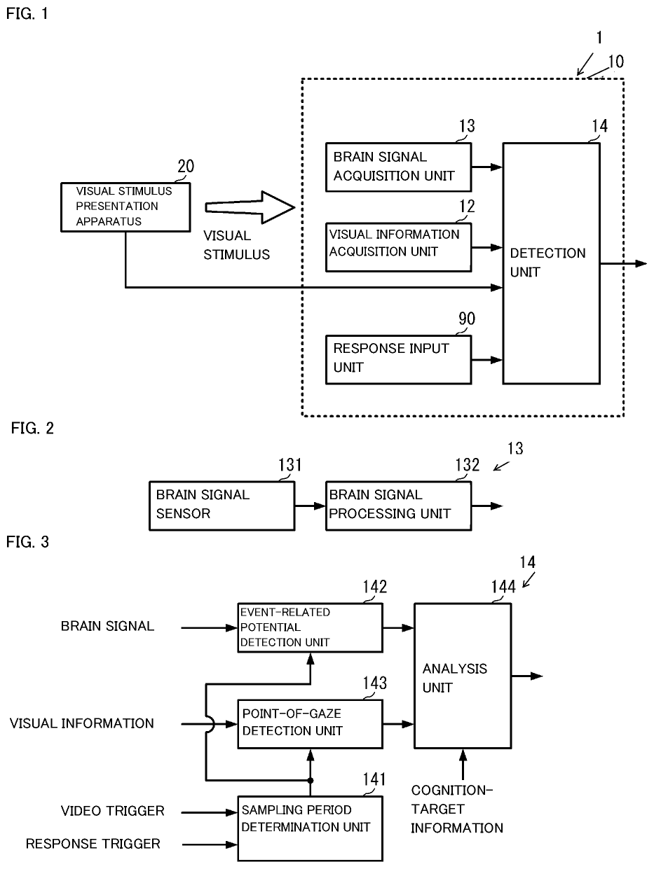

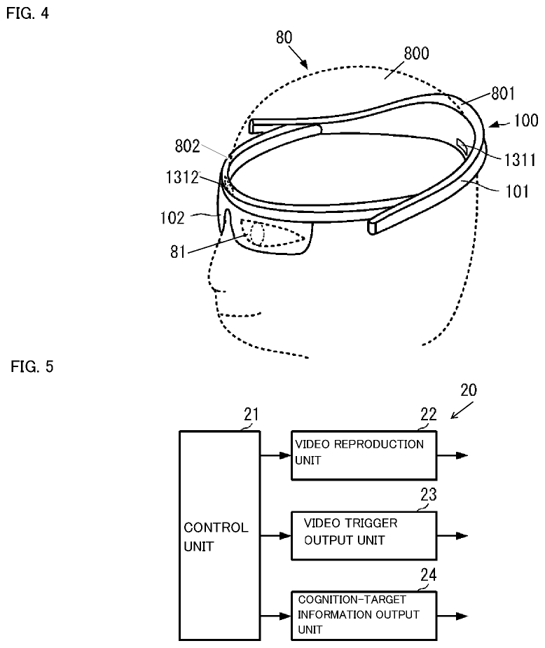

A cognitive ability detection apparatus according to a first embodiment of the present disclosure will be described with reference to the drawings. FIG. 1 is a functional block diagram of a cognitive ability detection system including the cognitive ability detection apparatus according to the first embodiment of the present disclosure. FIG. 2 is a functional block diagram illustrating a configuration of a brain signal acquisition unit. FIG. 3 is a functional block diagram illustrating a configuration of a detection unit. FIG. 4 is a diagram illustrating an overview of a state of a detection gear worn by a subject whose cognitive ability is to be detected. FIG. 5 is a functional block diagram illustrating a configuration of a visual stimulus presentation apparatus.

(Configuration of Schematic Functional Blocks)

As illustrated in FIG. 1, a cognitive ability detection system includes a cognitive ability detection apparatus 10 and a visual stimulus presentation apparatus 20. The cognitive ability detection apparatus 10 includes a visual information acquisition unit 12, a brain signal acquisition unit 13, a detection unit 14, and a response input unit 90. Note that the response input unit 90 may be omitted from the cognitive ability detection apparatus 10 in the case where a cognitive ability is detected by using P300 described later.

The visual information acquisition unit 12 is implemented by a known eye tracking sensor. The visual information acquisition unit 12 detects the eye movement of a subject 80 and outputs the information on the eye movement as visual information.

As illustrated in FIG. 2, the brain signal acquisition unit 13 includes a brain signal sensor 131 and a brain signal processing unit 132. The brain signal sensor 131 is implemented by, for example, a known sensor capable of acquiring a brain signal. The brain signal processing unit 132 is implemented by, for example, an electronic circuit, an IC, or the like. The brain signal sensor 131 acquires a brain signal of the subject 80 and outputs the brain signal to the brain signal processing unit 132. The brain signal processing unit 132 performs processing such as filtering and amplification on the brain signal acquired by the brain signal sensor 131, and outputs the resultant signal.

The response input unit 90 is implemented by, for example, a pseudo-steering wheel, a pseudo-brake pedal, a pseudo-brake lever, or the like in the case where a danger cognitive ability in driving an automobile is detected as the cognitive ability. That is, the response input unit 90 is implemented by a member capable of detecting an action of the subject 80 in response to the occurrence of a cognition-target event. The response input unit 90 generates a response trigger in synchronization with an input timing of a response of the subject 80 to a cognition-target event.

As illustrated in FIG. 3, the detection unit 14 includes a sampling period determination unit 141, an event-related potential detection unit 142, a point-of-gaze detection unit 143, and an analysis unit 144. Each of the units of the detection unit 14 is implemented by, for example, an electronic circuit, an IC, an MPU, or the like.

A video trigger from the visual stimulus presentation apparatus 20 or a response trigger from the response input unit 90 is inputted to the sampling period determination unit 141. Although details will be described later, the sampling period determination unit 141 determines a first sampling period based on the video trigger. The sampling period determination unit 141 determines a second sampling period based on the response trigger. The sampling period determination unit 141 outputs the first sampling period or the second sampling period to the event-related potential detection unit 142 and the point-of-gaze detection unit 143.

A brain signal is inputted to the event-related potential detection unit 142. The event-related potential detection unit 142 detects a specific event-related potential from the brain signal in the first sampling period or the second sampling period. The event-related potential detection unit 142 outputs the detected event-related potential to the analysis unit 144.

The visual information is inputted to the point-of-gaze detection unit 143. The point-of-gaze detection unit 143 detects a point-of-gaze position from the visual information in the first sampling period. The point-of-gaze detection unit 143 outputs the detected point-of-gaze position to the analysis unit 144.

The event-related potential, the point-of-gaze position, and cognition-target information are inputted to the analysis unit 144. The cognition-target information includes a position of a cognition target in video at the time of generation of the video trigger. The analysis unit 144 analyzes the cognitive ability of the subject 80 by using the event-related potential, the point-of-gaze position, and the cognition-target information. Based on the analysis result, the analysis unit 144 detects whether the subject 80 has the cognitive ability to the cognition target, the cognitive ability level, and so on.

(Configuration of Detection Gear 100)

A detection gear 100 such as one illustrated in FIG. 4 is equipped with some of the components of the cognitive ability detection apparatus 10. The detection gear 100 includes a headband 101 and a plate member 102. The headband 101 is made of a band-shaped base material. The headband 101 has, for example, an elastic property. The headband 101 is worn around a head 800 of the subject 80 whose cognitive ability is to be detected. More specifically, the headband 101 is worn by the subject 80 so as to be around the entire circumference of the head 800 including a back head portion 801, side head portions, and a front head portion 802 of the subject 80.

The headband 101 is equipped with a brain signal sensor 1311, which is on the inner side of the headband 101 at the back head portion 801. The headband 101 is equipped with a brain signal sensor 1312, which is on the inner side of the headband 101 at the front head portion 802. The brain signal sensor 1311 and the brain signal sensor 1312 acquire and output brain signals of the subject 80. The brain signal sensor 131 includes the brain signal sensor 1311 and the brain signal sensor 1312.

Since the headband 101 has an elastic property, the headband 101 is in close contact with the back head portion 801 and the front head portion 802 of the subject 80. Consequently, the brain signal sensor 1311 is in close contact with the back head portion 801 of the subject 80, and the brain signal sensor 1312 is in close contact with the front head portion 802 of the subject 80. Therefore, the brain signal sensor 1311 and the brain signal sensor 1312 easily acquire brain signals generated by the subject 80. The headband 101 may further include another brain signal sensor that is in contact (close contact) with a top head portion of the subject 80. It is sufficient that the headband 101 has at least one brain signal sensor.

The plate member 102 has a light-transmissive property. The plate member 102 is disposed near a portion of the headband 101 where the brain signal sensor 1312 is located. The plate member 102 has a shape protruding from the lower end of the headband 101. The plate member 102 has, for example, a shape similar to that of lenses of eyeglasses. The plate member 102 has a shape so that the plate member 102 overlaps with eyes 81 of the subject 80 when the subject 80 looks forward while wearing the headband 101. The plate member 102 is equipped with the above-described eye tracking sensor (not illustrated.).

(Configuration of Visual Stimulus Presentation Apparatus 20)

As illustrated in FIG. 5, the visual stimulus presentation apparatus 20 includes a control unit 21, a video reproduction unit 22, a video trigger output unit 23, and a cognition-target information output unit 24. The visual stimulus presentation apparatus 20 is implemented by, for example, an electronic circuit, an IC, an MPU, or the like, and a display that displays video.

The video reproduction unit 22 reproduces video including a visual stimulus for which the cognitive ability is to be detected, and displays the video on the display. The video trigger output unit 23 generates and outputs a video trigger at an occurrence timing of the visual stimulus in the video. The cognition-target information output unit 24 generates and outputs cognition-target information including the position of the visual stimulus (the position of the cognition target) in the video. The control unit 21 controls synchronization among the video reproduction unit 22, the video trigger output unit 23, and the cognition-target information output unit 24 and controls the entire visual stimulus presentation apparatus 20.

The cognitive ability detection apparatus 10 and the cognitive ability detection system 1 configured as described above detect the cognitive ability of the subject 80 in the following manner. A case of detecting the cognitive ability of the subject 80 to danger, more specifically, the danger cognitive ability in driving an automobile will be described below. Note that the configurations and processes of the present embodiment are applicable to other events as long as the cognitive ability is detected by using video.

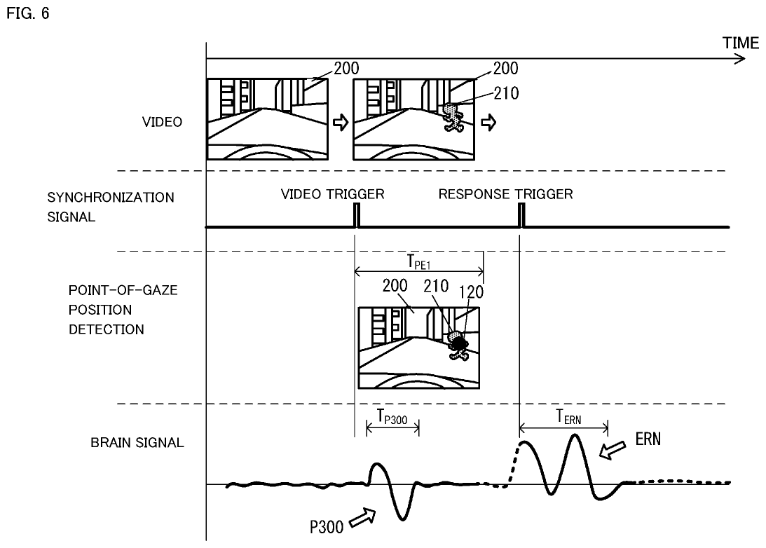

FIG. 6 is a diagram illustrating a concept of cognitive ability detection. In FIG. 6, video, a synchronization signal (trigger), a point-of-gaze position detection state, and a brain signal are schematically illustrated in accordance with an elapse of time.

First, the visual stimulus presentation apparatus 20 provides the subject 80 with dynamic video 200 including a visual stimulus serving as a danger cognition target. The video 200 includes a frame not including a danger cognition target 210 and a frame including the danger cognition target 210. Specifically, the danger cognition target 210 is, for example, a person model or the like that may enter a traveling path of the automobile from a side street.

The visual stimulus presentation apparatus 20 reproduces a frame not including the danger cognition target 210 and then reproduces a frame including the danger cognition target 210. The visual stimulus presentation apparatus 20 also outputs a video trigger in synchronization with the start of the reproduction of the frame including the danger cognition target 210. The visual stimulus presentation apparatus 20 further outputs the cognition-target information when the frame including the danger cognition target 210 is reproduced.

The visual information acquisition unit 12 of the cognitive ability detection apparatus 10 continuously detects eye movement of the subject 80 with respect to the video and outputs information on the eye movement as visual information. The point-of-gaze detection unit 143 detects a point-of-gaze position with respect to the video by using the visual information and outputs the detected point-of-gaze position. For example, the point-of-gaze detection unit 143 detects whether the point-of-gaze position coincides with the person model that may enter the traveling path of the automobile from the side street, and outputs the detection result. At this time, the point-of-gaze detection unit 143 continuously detects and outputs the point-of-gaze position.

The brain signal acquisition unit 13 of the cognitive ability detection apparatus 10 acquires and outputs brain signals of the subject 80 who is viewing the video. At this time, the brain signal acquisition unit 13 continuously acquires and outputs the brain signals.

(Detection of Danger Cognitive Ability by Using P300)

P300 is one of event-related potentials and occurs after approximately 300 msec. from when a stimulus (dangerous condition) is grasped. P300 is used to detect the cognitive ability of the subject 80 to a potential danger (stimulus). Although the case of using P300 as the event-related potential is described herein, P100 may be used, or both P100 and P300 may be used.

The sampling period determination unit 141 of the detection unit 14 determines the first sampling period in accordance with the video trigger. The sampling period determination unit 141 individually sets a sampling period T_{PEI} for detecting the point-of-gaze position and a sampling period T_{P300} for detecting P300 as the first sampling period. The sampling period T_{PEI} is set to have a predetermined time length from the video trigger that serves as a start timing (starting point). The sampling period T_{P300} is set to have a predetermined time length from a timing at which the point-of-gaze detection unit 143 detects that a point-of-gaze position 120 coincides with the position of the danger cognition target 210 and which serves as a start timing (starting point). Note that the sampling period T_{P300} may be set by using the video trigger as the start timing (starting point). The time length of the sampling period T_{P300} is, for example, 500 msec.

The event-related potential detection unit 142 detects P300 in the sampling period T_{P300} and outputs the detection result to the analysis unit 144. P300 is a signal having a specific waveform. The event-related potential detection unit 142 is able to detect P300 by using this waveform and the amplitude.

The point-of-gaze detection unit 143 detects the point-of-gaze position 120 in the video 200 in the sampling period T_{PEI}, and outputs the detection result to the analysis unit 144.

The analysis unit 144 analyzes the cognitive ability, based on the detection result of the point-of-gaze position 120 and the detection result of P300. For example, in the example illustrated in FIG. 6, the amplitude (output level) of P300 is more than a level recognizable as P300 and is large. The point-of-gaze position 120 coincides with the position of the danger cognition target 210. That is, there is an effective point of gaze for the danger cognition target 210. In this case, the analysis unit 144 detects that the cognitive ability level of the subject 80 to the potential danger is high or the subject 80 has the cognitive ability to the potential danger.

On the other hand, for example, when the amplitude of P300 is smaller than the recognizable level and the point-of-gaze position 120 is separate from the position of the danger cognition target 210 (when there is an ineffective point of gaze for the danger cognition target 210), the analysis unit 144 detects that the cognitive ability level of the subject 80 to the potentially danger is low or the subject 80 lacks the cognitive ability to the potentially danger.

For example, when the amplitude of P300 is more than or equal to the recognizable level and is large but the point-of-gaze position 120 is separate from the position of the danger cognition target 210, the analysis unit 144 may detect that the cognitive ability level of the subject 80 to the potential danger is low or the subject 80 has an issue in the cognitive ability to the potential danger.

For example, when the amplitude of P300 is smaller than the recognizable level but the point-of-gaze position 120 coincides with the position of the danger cognition target 210, the analysis unit 144 may detect that the subject 80 has the cognitive ability to the potential danger but awareness of the potential danger is low.

(Detection of Danger Cognitive Ability by Using ERN)

Error-related negativity (ERN) is one of event-related potentials and occurs when the subject 80 recognizes by himself/herself that a response of the subject 80 to a stimulus (dangerous state) is incorrect. ERN is used to detect the cognitive ability of the subject 80 to internally informed danger (stimulus).

The sampling period determination unit 141 of the detection unit 14 determines the second sampling period in accordance with a response trigger. The sampling period determination unit 141 sets a sampling period T_{ERN} for detecting ERN as the second sampling period. The sampling period T_{ERN} is set to have a predetermined time length from the response trigger.

The event-related potential detection unit 142 detects ERN in the sampling period T_{ERN} and outputs the detection result to the analysis unit 144. ERN is a signal having a specific waveform. The event-related potential detection unit 142 is able to detect ERN by using this waveform and the amplitude.

Based on the detection result of ERN, the analysis unit 144 analyzes the cognitive ability. For example, in the example of FIG. 6, the amplitude (output level) of ERN is more than a level recognizable as ERN and is large. If the response is incorrect, the analysis unit 144 detects that the subject 80 notices his or her incorrect response and that the cognitive ability level of the subject 80 to the internally informed danger is high or the subject 80 has the cognitive ability to the internally informed danger. If the response is correct and if no ERN is detected, the analysis unit 144 detects that the cognitive ability level of the subject 80 to the danger is high or the subject 80 has the cognitive ability to the danger.

On the other hand, for example, if the response is incorrect and the amplitude of ERN is smaller than the recognizable level, the analysis unit 144 detects that the subject 80 does not notice his or her incorrect response and that the cognitive ability level of the subject 80 to the internally informed danger is low or the subject 80 lacks the cognitive ability to the internally informed danger.

For example, if a response is made despite there being no danger and the amplitude of ERN is more than or equal to the recognizable level and is large, the analysis unit 144 detects that the cognitive ability level of the subject 80 to the internally informed danger is high or the subject 80 has the cognitive ability to the internally informed danger.

For example, if a response is made despite there being no danger and the amplitude of ERN is smaller than the recognizable level, the analysis unit 144 detects that the cognitive ability level of the subject 80 to the internally informed danger is low or the subject 80 lacks the cognitive ability to the internally informed danger.

For example, if there is no danger, there is no response, and the amplitude of ERN is smaller than the recognizable level, the analysis unit 144 detects that the cognitive ability level of the subject 80 to the internally informed danger is high or the subject 80 has the cognitive ability to the internally informed danger.

As described above, the use of the configurations and processes of the present embodiment enables the cognitive ability of the subject 80 to visually presented danger to be detected more accurately and reliably than in the related art.

In the description above, the case of detecting the cognitive ability to the potential danger by using P300 and the case of detecting the cognitive ability to the internally informed danger by using ERN are presented separately. However, the danger cognitive ability may be detected collectively by using P300 and ERN. Alternatively, the danger cognitive ability may be detected by using P300 alone or ERN alone.

(Cognitive Ability Detection Method)

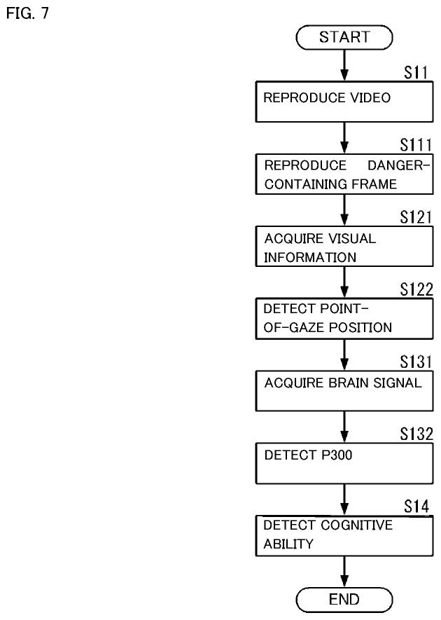

In the description above, the case of detecting the cognitive ability by using the plurality of functional units has been presented. However, a program of a process illustrated in FIG. 7 may be stored and executed by an arithmetic processing device such as a CPU. FIG. 7 is a flowchart of a cognitive ability detection method using P300 according to the first embodiment of the present disclosure. The specific content of each processing is described above. Thus, specific description will be omitted below.

The arithmetic processing device reproduces video for the subject 80 (S11). The arithmetic processing device reproduces video including a visual stimulus (danger-containing frame) (S111). The arithmetic processing device acquires visual information (S121) and detects a point-of-gaze position in the first sampling period (S122). The arithmetic processing device acquires a brain signal (S131) and detects P300 in the first sampling period (S132). The arithmetic processing device detects the cognitive ability by using the cognition-target information, the point-of-gaze position, and P300 (S14).

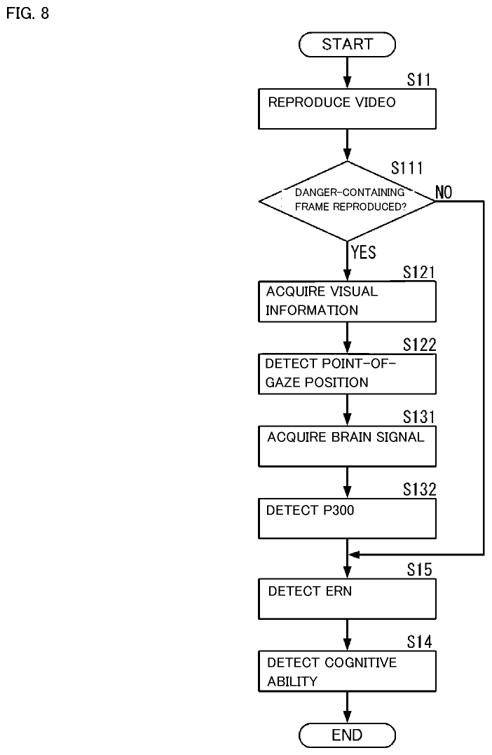

FIG. 8 is a flowchart illustrating a cognitive ability detection method using P300 and ERN according to the first embodiment of the present disclosure. The processing that involves P300 in FIG. 8, that is, the processing that involves the first sampling period is the same as that in FIG. 7. Thus, the description of the same processing is omitted.

When reproducing the danger-containing frame (S111: YES), the arithmetic processing device performs detection by using P300 as in FIG. 7. On the other hand, when not reproducing the danger-containing frame (S111: NO), the arithmetic processing device performs detection processing by using ERN (S15).

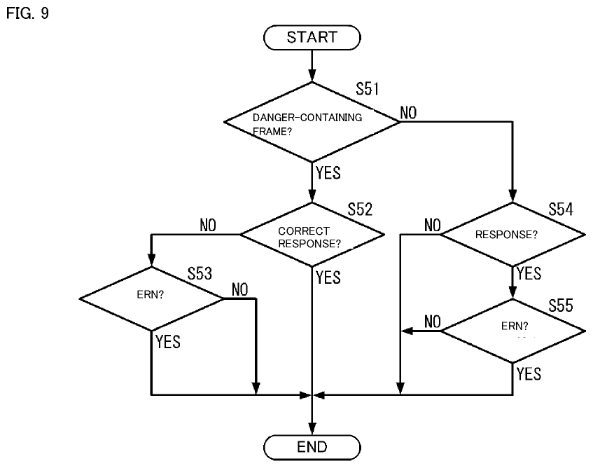

FIG. 9 is a flowchart illustrating an example of a cognitive ability detection method using ERN.

If there is a danger-containing frame (S51: YES) and the response is correct (S52: YES), the arithmetic processing device detects that the cognitive ability level is high or the cognitive ability is present. Note that the term “response” in this case refers to, for example, performing a danger avoidance action such as braking for deceleration or an appropriate steering operation in the case where the cognitive ability detection apparatus of the present embodiment is used as a training simulator at a driving school.

If there is a danger-containing frame (S51: YES) and the response is incorrect (S52: NO), the arithmetic processing device performs detection of ERN. If ERN is successfully detected (S53: YES), the arithmetic processing device detects that the cognitive ability level is high or the cognitive ability is present. On the other hand, if ERN is not detected (S53: NO), the arithmetic processing device detects that the cognitive ability level is low or the cognitive ability is absent.

If the reproduced frame is not a danger-containing frame (S51: NO) and there is no response (S54: NO), the arithmetic processing device detects that the cognitive ability level is high or the cognitive ability is present. At this point, the arithmetic processing device may suspend the determination of the cognitive ability.

If the reproduced frame is not a danger-containing frame (S51: NO) and there is a response (S54: YES), the arithmetic processing device performs detection of ERN. If ERN is successfully detected (S55: YES), the arithmetic processing device detects that the cognitive ability level is high or the cognitive ability is present. On the other hand, if ERN is not detected (S55: NO), the arithmetic processing device detects that the cognitive ability level is low or the cognitive ability is absent.

The arithmetic processing device detects the cognitive ability by using the detection result of the cognitive ability based on P300 and the detection result of the cognitive ability based on ERN.

In FIG. 8, by skipping the processing from step S111 to step S15, the arithmetic processing device is able to detect the cognitive ability based on ERN alone.

The use of the cognitive ability detection apparatus according to the first embodiment enables the cognitive ability of the subject of detection performed by the cognitive ability detection apparatus to be measured. For example, in the case where the cognitive ability detection apparatus according to the present embodiment is used as a training simulator at a driving school, an instructor is able to scientifically grasp the danger cognitive ability of a trainee. Thus, the instructor is able to give appropriate feedback to the trainee after the end of training.

### Second Embodiment

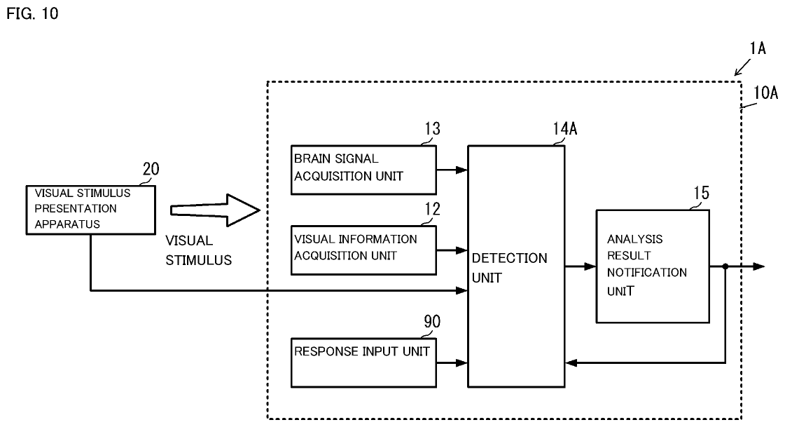

A cognitive ability detection apparatus according to a second embodiment of the present disclosure will be described with reference to the drawings. FIG. 10 is a functional block diagram of a cognitive ability detection system including the cognitive ability detection apparatus according to the second embodiment of the present disclosure.

As illustrated in FIG. 10, a cognitive ability detection system 1A according to the second embodiment differs from the cognitive ability detection system 1 according to the first embodiment in the configuration of a cognitive ability detection apparatus 10A. The cognitive ability detection apparatus 10A further includes an analysis result notification unit 15 compared with the cognitive ability detection apparatus 10. The cognitive ability detection apparatus 10A differs in processing performed by a detection unit 14A. Other configurations and processes of the cognitive ability detection system 1A are substantially the same as those of the cognitive ability detection system 1, and the description of the same portions is omitted.

As illustrated in FIG. 10, the cognitive ability detection system 1A includes the cognitive ability detection apparatus 10A. The cognitive ability detection apparatus 10A includes the detection unit 14A and the analysis result notification unit 15.

The analysis result notification unit 15 makes a notification according to the cognitive ability detected by the detection unit 14A. For example, the analysis result notification unit 15 includes a sound output unit (not illustrated.) and makes a notification about the detection result of the cognitive ability by sound 230S. The sound output unit can be implemented by, for example, a speaker attached to the headband 101. The analysis result notification unit 15 may make a notification about the detection result of the cognitive ability by a marking 230V. In this case, the analysis result notification unit 15 outputs the marking 230V to the visual stimulus presentation apparatus 20. The visual stimulus presentation apparatus 20 reproduces the video on which the marking 230V is superimposed.

The analysis result notification unit 15 generates a notification trigger at the notification timing and outputs the notification trigger to the detection unit 14A.

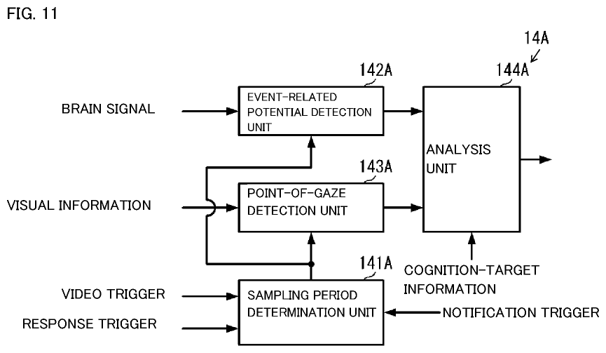

FIG. 11 is a functional block diagram illustrating a configuration of the detection unit 14A. As illustrated in FIG. 11, the detection unit 14A includes a sampling period determination unit 141A, an event-related potential detection unit 142A, a point-of-gaze detection unit 143A, and an analysis unit 144A.

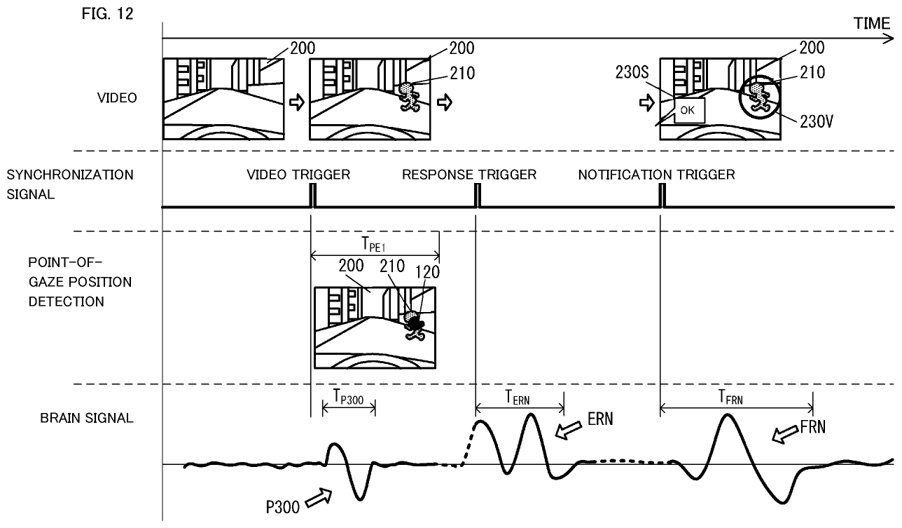

As with the detection unit 14 in the first embodiment, the detection unit 14A detects the cognitive ability by using P300 and ERN. The detection unit 14A also detects the cognitive ability by using feedback-related negativity (FRN) as described below. FIG. 12 is a diagram illustrating a concept of cognitive ability detection. In FIG. 12, as in FIG. 6, video, a synchronization signal, a point-of-gaze position detection state, and a brain signal are schematically illustrated in accordance with an elapse of time.

(Detection of Danger Cognitive Ability by Using FRN)

FRN is one of event-related potentials and occurs when the subject 80 recognizes that a response of the subject 80 to a stimulus (dangerous state) is incorrect as a result of the incorrectness being pointed out by others. FRN is used to detect the cognitive ability of the subject 80 to the externally informed danger (stimulus).

The sampling period determination unit 141A of the detection unit 14A determines a third sampling period in accordance with a notification trigger. The sampling period determination unit 141A sets a sampling period T_{ERN} for detecting FRN as the third sampling period. For example, the sampling period T_{ERN} is set to have a predetermined time length from the notification trigger that serves as a start timing.

The event-related potential detection unit 142A detects FRN in the sampling period T_{ERN} and outputs the detection result to the analysis unit 144A. FRN is a signal having a specific waveform. The event-related potential detection unit 142A is able to detect FRN by using this waveform and the amplitude.

Based on the detection result of FRN, the analysis unit 144A analyzes the cognitive ability. For example, in the example of FIG. 12, the amplitude (output level) of FRN is more than a level recognizable as FRN and is large. In this case, the analysis unit 144A detects that the cognitive ability level of the subject 80 to the externally informed danger is high or the subject 80 has the cognitive ability to the externally informed danger.

On the other hand, for example, when the amplitude (output level) of FRN is smaller than the recognizable level, the analysis unit 144A detects that the cognitive ability level of the subject 80 to the externally informed danger is low or the subject 80 lacks the cognitive ability to the externally informed danger.

When the analysis unit 144A uses the marking 230V as the notification, the analysis unit 144A may detect the cognitive ability in consideration of the point-of-gaze position in the third sampling period as well as FRN. For example, if the marking 230V coincides with the point-of-gaze position, the analysis unit 144A can use this as a reference to obtain the detection result indicating that the cognitive ability level is high or the cognitive ability is present.

As described above, the use of the configurations and processes of the present embodiment enables the externally assisted cognitive ability of the subject 80 to visually presented danger to be detected. The use of the configurations and processes of the present embodiment also enables the cognitive ability of the subject 80 to visually presented danger to be detected more accurately and reliably than in the related art.

In the description above, the case of detecting the cognitive ability to danger by using P300, ERN, and FRN is presented. However, the cognitive ability to danger may be detected by using FRN alone. The cognitive ability to danger may also be detected by using P300 and FRN but not using ERN, or by using ERN and FRN but not using P300.

The use of the configuration and process for making a notification about an analysis result as described in the second embodiment enables training of the cognitive ability to be performed through repeated reproduction of video and repeated detection of the cognitive ability. That is, training for improving the cognitive ability of the subject 80 can be implemented by repeatedly detecting the cognitive ability while feeding back the detection result of the cognitive ability to the subject 80. In this manner, a neurofeedback system for a visual stimulus can be implemented.

At this time, detection of the cognitive abilities for the individual danger types enables implementation of more effective training. Specifically, for example, in the case of the above-described driving simulator, the danger types are classified based on visually different positions or different phenomena such as jumping out of a person or a stimulus on a rearview mirror. If the cognitive ability to specific danger is absent or low, training is repeatedly performed mainly on this specific danger. Specifically, when neurofeedback is performed, the danger cognitive level based on the brain signals of the subject 80 who is a trainee is desirably fed back visually or auditorily in real time after the stimulus is presented. Based on the feedback, the subject 80 can devise the training method and make an effort to improve the danger cognitive level. In this manner, a more effective training system than a system in which a trainee is just trained without feedback can be implemented.

According to the training based on the second embodiment, a higher training effect can be obtained than that obtained with the feedback described in the first embodiment (for example, feedback given by the instructor to the trainee after the end of the training).

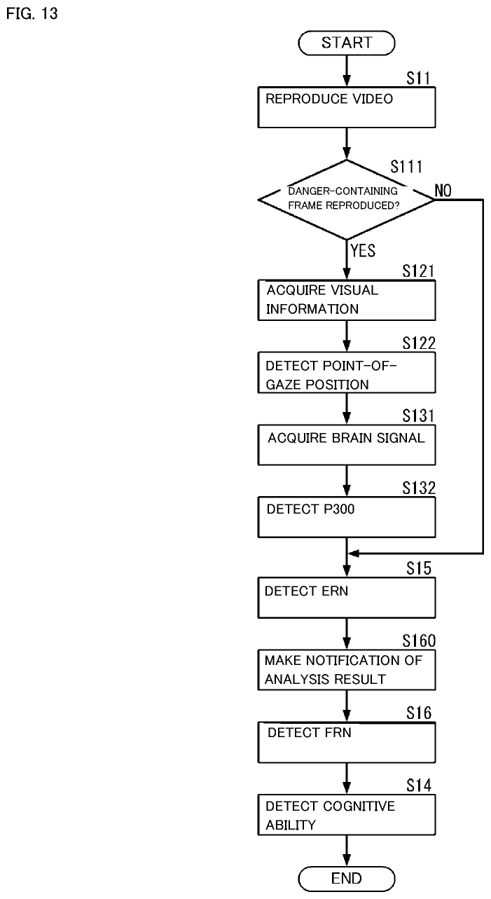

FIG. 13 is a flowchart of a cognitive ability detection method using P300, ERN, and FRN according to the second embodiment of the present disclosure. The processing that involves P300 and ERN in FIG. 13, that is, the processing that involves the first sampling period and the second sampling period is the same as that in FIG. 8. Thus, the description of the same processing is omitted.

After detecting ERN in the second sampling period, the arithmetic processing device makes a notification about the analysis result (S160).

The arithmetic processing device sets the third sampling period from the notification trigger that serves as a starting point. The arithmetic processing device detects FRN in the third sampling period (S16).

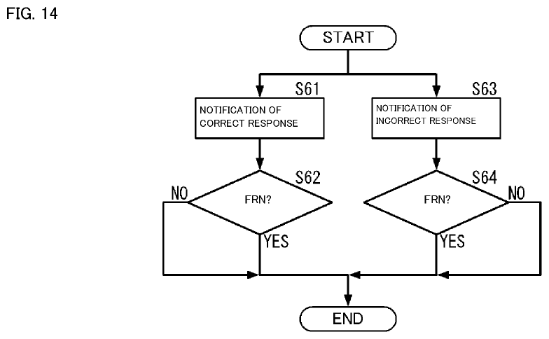

FIG. 14 is a flowchart of an example of a cognitive ability detection method using FRN.

In response to making a notification indicating that the response is correct (S61), the arithmetic processing device performs detection of FRN. If the subject 80 understands the correctness and the arithmetic processing device successfully detects FRN (S62: YES), the arithmetic processing device detects that the cognitive ability level is high or the cognitive ability is present. On the other hand, if the subject 80 fails to understand the correctness and the arithmetic processing device does not detect FRN (S62: NO), the arithmetic processing device detects that the cognitive ability level is low or the cognitive ability is absent.

In response to making a notification indicating that the response is incorrect (S63), the arithmetic processing device performs detection of FRN. If the subject 80 understands the incorrectness and the arithmetic processing device successfully detects FRN (S64: YES), the arithmetic processing device detects that the cognitive ability level is high or the cognitive ability is present. On the other hand, if the subject 80 fails to understand the incorrectness and the arithmetic processing device does not detect FRN (S64: NO), the arithmetic processing device detects that the cognitive ability level is low or the cognitive ability is absent.

The arithmetic processing device detects the cognitive ability by using the detection result obtained using P300, the detection result obtained using ERN, and the detection result obtained using FRN.

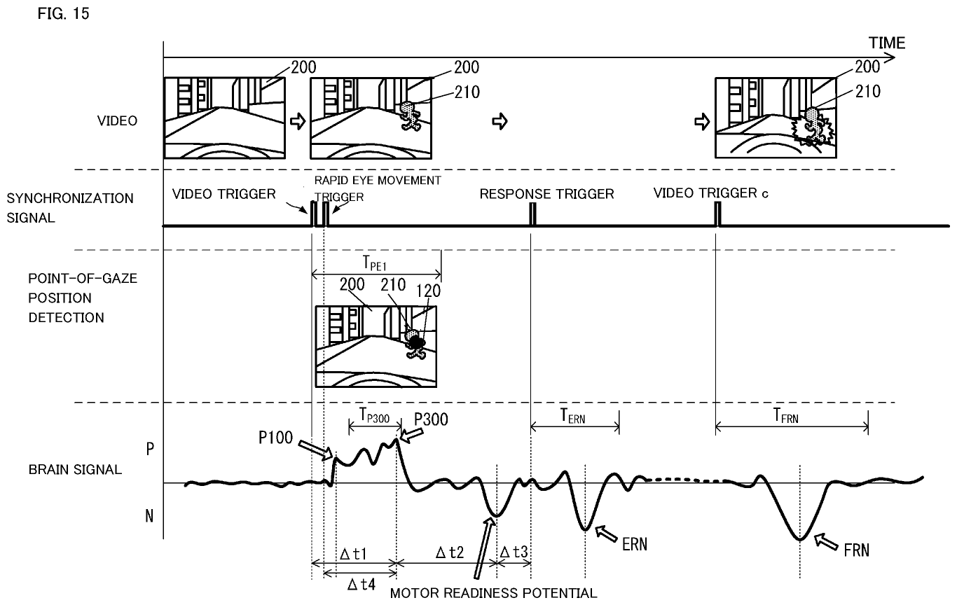

In addition to the above-described detection of the cognitive ability by using P300, ERN, and FRN, the cognitive ability detection apparatus may detect the cognitive ability by using the following items. FIG. 15 is a diagram illustrating a concept of cognitive ability detection. In FIG. 15, as in FIG. 12, video, a synchronization signal, a point-of-gaze position detection state, and a brain signal are schematically illustrated in accordance with an elapse of time. In FIG. 15, each event-related potential is expressed on a coordinate axis including a positive region and a negative region. Note that these are merely examples, and the waveform of each event-related potential is not limited to this. In addition, a case will be described below in which analysis is performed with the configuration of the detection unit 14A illustrated in FIG. 11 described above. However, when FRN is not used, analysis can be performed with the configuration of the detection unit 14.

(Example of Detection of Cognitive Ability by Using Motor Readiness Potential)

The motor readiness potential is one of the event-related potentials, and is a signal that occurs in a preparation period from when P300 occurs to when a danger avoidance action is started by using a steering wheel, a brake pedal, a brake lever, or the like in the case of the driving simulator described above, for example. The motor readiness potential can be detected from the brain signal similarly to P300, ERN, FRN, and the like.

The analysis unit 144A analyzes and evaluates a cognitive speed to be ready for the danger avoidance action from a time difference Δt2 between a detection timing of P300 and a detection timing of the motor readiness potential. For example, the analysis unit 144A evaluates that the cognitive speed to be ready for the danger avoidance action is fast when the time difference Δt2 is short, and evaluates that the cognitive speed to be ready for the danger avoidance action is slow when the time difference Δt2 is long.

The analysis unit 144A may perform other types of analysis and evaluation by using the detection timing of each event-related potential and the detection timing (acquisition timing) of each trigger. The detection timing of each event-related potential can be defined by, for example, the time of the maximum value or the minimum value in the sampling period of the event-related potential. For example, as in the case of FIG. 15, the detection timing of ERN is the timing at which ERN takes the minimum value, and the detection timing of FRN is the timing at which FRN takes the minimum value.

For example, the analysis unit 144A is able to analyze and evaluate an initial cognitive speed from a time difference Δt1 between a detection timing of a video trigger and the detection timing of P300. The analysis unit 144A is also able to analyze and evaluate an action start speed of a danger avoidance action from a time difference Δt3 between the detection timing of the motor readiness potential and a detection timing of a response trigger.

If the point-of-gaze detection unit 143A is capable of obtaining a rapid eye movement trigger, the analysis unit 144A is able to detect the cognitive ability in accordance with the rapid eye movement trigger. The rapid eye movement is obtained by the aforementioned eye tracking sensor or an electrooculography detection sensor. For example, in the case of the eye tracking sensor, the rapid eye movement can be detected by calculating a movement speed of the point-of-gaze position. The analysis unit 144A is able to analyze and evaluate the cognitive speed and the like from a time difference Δt4 between the detection timing of the rapid eye movement trigger and the detection timing of each event-related potential.

The analysis unit 144A is able to analyze and evaluate the danger cognitive ability of the subject 80 in more detail by using the rapid eye movement trigger. For example, the analysis unit 144A acquires the point-of-gaze position at the detection timing of the rapid eye movement trigger. The analysis unit 144A compares this point-of-gaze position with the position of the visual stimulus described above. In this manner, the analysis unit 144A is able to detect whether the eye movement for danger cognition is correct, that is, whether the subject can correctly recognize the danger. Specifically, for example, if the point-of-gaze position at the detection timing of the rapid eye movement trigger coincides with the position of the visual stimulus, the analysis unit 144A detects that the cognitive ability is high. If the point-of-gaze position at the detection timing of the rapid eye movement trigger is separate greatly from the position of the visual stimulus, the analysis unit 144A detects that the cognitive ability is absent.

In the above-described method using the timings, the case of using P300 as the event-related potential is presented. However, also in this case, P100 may be used as the event-related potential. Furthermore, both P300 and P100 may be used as event-related potentials.

In FIG. 15, instead of using the above-described notification to the response, video presenting a danger occurrence event is used. That is, a video trigger c in this video is used. Analysis and evaluation can be performed based on FRN by using video presenting such a danger occurrence event.

FIG. 15 illustrates the case of using both the detection timing of the rapid eye movement trigger and the detection timing of the motor readiness potential. However, the case of using one of these timings may be possible.
1, 1A: cognitive ability detection system
10, 10A: cognitive ability detection apparatus
12: visual information acquisition unit
13: brain signal acquisition unit
14, 14A: detection unit
15: analysis result notification unit
20: visual stimulus presentation apparatus
21: control unit
22: video reproduction unit
23: video trigger output unit
24: cognition-target information output unit
80: subject
81: eye
90: response input unit
100: detection gear
101: headband
102: plate member
120: point-of-gaze position
131: brain signal sensor
132: brain signal processing unit
141, 141A: sampling period determination unit
142, 142A: event-related potential detection unit
143, 143A: point-of-gaze detection unit
144, 144A: analysis unit
200: video
210: cognition target
230S: sound
230V: marking
800: head
801: back head portion
802: front head portion
1311, 1312: brain signal sensor

1. A cognitive ability detection apparatus comprising:
an eye tracking sensor configured to acquire eye movement information of a subject in response to a visual stimulus in a video;
a brain signal sensor configured to acquire a brain signal of the subject; and
at least one circuit configured to:
detect an event-related potential from the brain signal based on a video trigger corresponding to a timing of the visual stimulus,
detect a point of gaze for the visual stimulus from the eye movement information, and
detect a cognitive ability level to the visual stimulus based on the event-related potential and/or the point of gaze.

2. The cognitive ability detection apparatus according to claim 1,
wherein the event-related potential comprises a P300 potential, and
wherein the at least one circuit is configured to detect the cognitive ability level based on an output level of the P300 in a first sampling period, and based on whether the point of gaze is at a position of the visual stimulus, the first sampling period being based on the video trigger.

3. The cognitive ability detection apparatus according to claim 1,
wherein the event-related potential comprises an error-related negativity (ERN) potential,
wherein the cognitive ability detection apparatus further comprises a response input device configured to receive a response of the subject to the video, and
wherein the at least one circuit is configured to detect the cognitive ability level based on an output level of the ERN in a second sampling period, the second sampling period being based on a response trigger corresponding to an input timing of the response.

4. The cognitive ability detection apparatus according to claim 1,
wherein the event-related potential comprises a feedback-related negativity (FRN) potential,
wherein the cognitive ability detection apparatus further comprises an output device configured to provide the subject with a notification about the detected cognitive ability level, and
wherein the at least one circuit is configured to detect the cognitive ability level based on an output level of the FRN in a third sampling period, the third sampling period being based on a notification trigger corresponding to a timing of the notification.

5. The cognitive ability detection apparatus according to claim 1,
wherein the visual stimulus comprises a danger cognition target, and
wherein the cognitive ability level is a danger cognitive ability level.

6. The cognitive ability detection apparatus according to claim 1,
wherein the event-related potential comprises a motor readiness potential, and
wherein the at least one circuit is configured to detect a cognitive speed as the cognitive ability level based on the motor readiness potential.

7. The cognitive ability detection apparatus according to claim 5,
wherein the event-related potential comprises a motor readiness potential, and
wherein the at least one circuit is configured to detect a cognitive speed as the cognitive ability level based on the motor readiness potential, the cognitive speed being a speed to be ready for a danger avoidance action.

8. The cognitive ability detection apparatus according to claim 1, wherein the at least one circuit is further configured to detect rapid eye movement from the eye movement information, and to detect the cognitive ability level based on the rapid eye movement.

9. The cognitive ability detection apparatus according to claim 1,
wherein the brain signal sensor is on or in a headband worn around a head of the subject.

10. The cognitive ability detection apparatus according to claim 9,
wherein the headband comprises a plate having a light-transmissive property, and
wherein the eye tracking sensor is on or in the plate.

11. The cognitive ability detection apparatus according to claim 1,
wherein the visual stimulus comprises a visual stimulus indicating danger in driving an automobile, and
wherein the at least one circuit is configured to detect, as the cognitive ability level, a danger cognitive ability level of the subject in driving an automobile.

12. A cognitive ability detection system comprising:
the cognitive ability detection apparatus according to claim 1; and
a display configured to display the video including the visual stimulus.
